(19) United States
(12) Patent Application Publication
(10) Pub. No.: US 2012/0245343 A1
(43) Pub. Date: Sep. 27, 2012

(54) ARGININE DERIVATIVES OR SALTS THEREOF EXHIBITING THE EFFECTS OF IMPROVING SEXUAL FUNCTION, COMPOSITION FOR IMPROVING SEXUAL FUNCTION COMPRISING SAME, AND METHOD FOR PREPARING SAME

(76) Inventor: Hae Dong Jang, Daejeon (KR)

(21) Appl. No.: 13/505,868

(22) PCT Filed: Oct. 15, 2010
(86) PCT No.: PCT/KR2010/007082

§ 371 Date: Jun. 12, 2012

(30) Foreign Application Priority Data
Nov. 3, 2009 (KR) 10-2009-0105303

(51) Int. Cl. C07H 7/02; C07H 1/00; C12P 19/26

(52) U.S. Cl. 536/55; 435/84

(57) ABSTRACT

The present invention provides a composition for improving sexual function comprising at least one compound as an active ingredient selected from the group consisting of arginine derivatives or pharmaceutically acceptable salts thereof. The composition of the present invention does not act as a substrate for arginase, but increases the generation of nitric oxide in endothelial cells along with enhancing the expression of nitric oxide synthase and induces the increase of the content of cGMP related to an erection depending on the concentration, thus remarkably exhibiting the effects of improving sexual function.

### TECHNICAL FIELD

The present invention relates to arginine derivatives or salts thereof exhibiting the effects of improving sexual function, a composition comprising the same and a method for manufacturing the same, more particularly to arginine derivatives or salts thereof that do not act as substrates for arginase, but increase the generation of nitric oxide in endothelial cells along with enhancing the expression of nitric oxide synthase, and induce the increase of the content of cGMP related to an erection depending on the concentration.

In addition, the present invention relates to a composition, for example, a medicine and a health functional food for improving sexual function comprising an arginine derivative or a salt thereof as an active ingredient.

Further, the present invention related to a method for manufacturing the aforesaid arginine derivatives or salts thereof by using food materials from animals and plants which are high in arginine and inducing the reaction between the sugar and the arginine contained in the food materials under a physicochemical condition.

### BACKGROUND ART

An erectile dysfunction is known as the most common sexual dysfunction. In general, the erectile dysfunction is caused by a psychological factor, a physiological factor or the combination of the two factors. Recently, the physiological factor is understood as a main factor which causes the erectile dysfunction. Therefore, the physiological mechanism of penile erection has been studied to treat the sexual dysfunction of the erectile dysfunction. The erection is a phenomenon that the corpora cavernosa are filled with blood by the relaxation of their arteries and then the blood can not escape from the corpora cavernosa by the relaxation of smooth muscle and the venous pressure when a man receives sexual stimulation.

It has been known that nitric oxide (NO) is involved in the erection and is formed in the presence of calcium, NADPH and calmodulin by using L-arginine as a precursor. A substance for generating nitric oxide binds to a receptor in the membrane of endothelial cells and increases the concentration of intracellular free calcium in the endothelial cells. As a result, the increased calcium enhances the synthesis and release of the nitric oxide. L-arginine, the precursor of nitric oxide present in all organisms, is a conditional essential amino acid. In the liver, the synthesis process of urea happens to remove ammonia in a human body, and arginine is decomposed into urea in urea cycle at that time. Arginine is necessary for generating epithelial cells, nerve cells, neutrophils, and nitric oxide. Foods containing a lot of arginine include meat, fish, dairy products, nuts and chocolate, etc. Meanwhile, the free nitric oxide enters into the smooth muscle cells of the corpus cavernosa and increases the production of cGMP by activating the guanylate cyclase of the smooth muscle cells. The increase in cGMP level leads to the erection of penis by the relaxation of the smooth muscle.

The erectile dysfunction can occur in all age groups and is a relatively common disease. Typically, it has been known that 5-10% of men suffers from the erectile dysfunction, but it is assumed that there are more patients. In south Korea, patients with the erectile dysfunction are continuously increasing due to dietary changes, average life expectancy, increased industrial accidents and traffic accidents, continuous physical fatigue and mental stress resulted from the complexities of modern life, etc. It is estimated by the Korean medical community that about 2.5-3 million people are suffering from the erectile dysfunction in south Korea nationwide. Thus, the development of the method and therapeutic agent for treating the erectile dysfunction is very important.

The therapeutic agent for treating the erectile dysfunction currently marketed is largely divided into three types by its mechanism of action. First, it is an inhibitor of an enzyme called phosphodiesterase-5 (PDE-5) present in erectile tissues, for example, Viagra® (sildenafil citrate), Levitra® (vardenafil) or Cialis® (tadalafil). Second, it is an agonist of prostaglandin E1 to expand blood vessels by directly acting on erectile tissues, for example, MUSE®, Befer® or Caverject® including alprostadil as an active ingredient. Third, it is an agonist of dopamine acting on D1/D2 dopamine receptors present in brains, for example, Urprima® including apomorphine as as a main ingredient. Additionally, it includes an antagonist of α-adrenoceptor, a stimulator of guanylate cyclase, and an agonist of serotonin (5-hydroxytryptamine) receptor.

However, it has been reported that most of the therapeutic agents for treating the erectile dysfunction developed until now show serious side effects and it is required to use them with the special attention. In addition, the therapeutic agents are classified into medical substances required for taking special care since there is a problem of abuse and misuse of them. For example, Viagra® for treating the erectile dysfunction by inhibiting PDE-5 shows side effects in patients with heart disease, such as visual impairment and hyperglycaemia, etc. Levitra® can lead to side effects such as headache, nausea, and facial flushing, etc. even with the difference of the degree of the side effects. Especially, if patients having angina and taking nitrates for angina treatment use Levitra®, there is a risk to decrease blood pressure rapidly in the patients.

Accordingly, it is more beneficial to develop an agent for preventing and treating sexual dysfunction using herbal materials and food materials that show the relatively low side effects. As reported to date, the Korean Rural Development Administration and Kun Wha Pharmaceutical Co., Ltd. have jointly developed “Nuegra™” and launched it on the market in 2001. This product is manufactured by extracting a male silkworm moth pupa and adding Acanthopanax senticosus, Schisandra chinensis, Rubus coreanus, royal jelly, Lycium chinense, etc. to the extract. Meanwhile, the Korean Institute of Medical Science and the medical school of the Inha University have jointly developed a herbal medicine of “Cheonbo 204™” for enhancing sexual function and launched it on the market. The Cheonbo 204™ is maunfactured from natural herbal materials such as red ginseng (Panax ginseng), Rubus coreanus, Schisandra chinensis, Lycium chinense, Cuscuta chinensis, Cornus officinalis, etc. In addition, the Korea Ginseng Corporation has developed “Red Max™” and launched it on the market, which includes red ginseng (Panax ginseng), Lycium chinense, Rubus coreanus, Schisandra chinensis, Torilis japonica, Cuscuta chinensis, etc. as main ingredients. Further, the Korea Food Research Institute and Andromedics Co. Ltd. have jointly developed a health functional food of “N40™” for improving male sexual function and launched it on the market, which is manufactured by extracting 12 species of natural substances such as Rubus coreanus, Cornus officinalis, Schisandra chinensis, Lycium chinense, Poria cocas, Astragalus membranaceus, Torilis japonica, Cuscuta chinensis, etc., and mixing enzymes to the extract.

With regard to the aforesaid health functional foods for improving sexual function which use ginseng or red ginseng resources, Korean Patent Publication No. 10-2004-0092232 disclosed a complex composition containing raw materials of red ginseng, natural herbal raw materials such as Lycium chinens, Rubus coreanus, Cuscuta chinensis, Schisandra chinensis and Torilis japonica, deer antlers, and health funtional raw materials such as odorless garlic, octacosanol, palatinose and L-carnitine. In addition, Korean Patent Publication No. 10-0850503 disclosed a composition for improving sexual function containing a complex extract of herbal materials such as Cornus officinalis, Schisandra chinensis, Acanthopanax senticosus, Rubus coreanus, red ginseng, garlic, Cinnamomum cassia, Cuscuta chinensis, Eucommia ulmoides and dried Rehmannia glutinosa as active ingredients. Further, Korean Patent Publication No. 10-2008-0100145 disclosed a composition for improving male sexual function (L-arginine being used simultaneously) containing ginseng fruit extract. From ancient times, ginseng or red ginseng has been used as a tonic nourishment and many studies have been performed until now, however, its effectiveness and pharmacological mechanisms have not been completely proven yet. It was merely reported that ginsenosides of ginseng saponins affect the generation of nitric oxide that plays an important role in male penile erection.

Besides, the prior arts also provided therapeutic agents for treating erectile dysfunction using L-arginine that is a precursor of nitric oxide as described above. For example, Korean Patent Publication No. 10-1996-0030945 disclosed a therapeutic agent for treating male sexual dysfunction containing L-arginine as a main component and prostaglandin E1, lecithin, tocopherol acetate, or a glutathione as a supplement ingredient. In addition, a transdermal formulation for treating erectile dysfunction and anorgasmia containing L-arginine was disclosed in Korean Patent Publication No. 10-2003-0041433.

Further, a medicine for improving sexual function has been developed and launched, which uses L-arginin together with medicinal herbals in the art. For example, a composition for preventing and treating male erectile dysfunction containing vegetable worms and Cornus officinalis as active ingredients was disclosed in Korean Patent Publication No. 10-2003-0049063, and a natural composition for treating sexual dysfunction consisting of L-arginine, ginseng and jujube (Chinese date) was also disclosed in Korean Patent Publication No. 10-2001-0074683.

As previously mentioned, various studies of health foods for showing the effects of improving sexual function are being performed in research laboratories of Universities and bio-venture companies, however, most of the conventional health foods merely use known effects of arginine itself or are manufactured simply by extracting natural herbs and mixing the extracts. The conventional health foods are sold by advertising their known effects, however, they cannot say that which of ingredients shows the effects of improving sexual function. In other words, the conventional health foods remain within the mixing of oriental herbal extracts, often known as a secret prescription in the oriental medical field.

It is noted that the conventional medicines for improving sexual function cause various side effects in a human body and most of the natural health foods for improving sexual function vaguely suggest the effects resulted from complex extracts of natural herbs based on the studies of the early stage without any concrete research about the relevant active ingredients.

In order to solve the aforesaid problems, it needs in the art to develop a medicine for improving sexual function without side effects in a human body by using natural materials, and to provide a technique for commercially manufacturing it and revealing active ingredients therein.

The inventor of the present invention had an interest in nutritional fortification of some natural materials such as ginseng and garlic, etc. and continued to study the tonic nourishment effects thereof. As a result, the inventor of the present invention found that arginine is very high in those natural materials and Amadori compound can be produced as a brown substance by amino-carbonyl reaction when processing those natural materials in accordance with the present invention.

It has been reported that arginine derivatives of arginyl-fructose (hereinafter, referred to as “AF”) and arginyl-fructosyl-glucose (hereinafter, referred to as “AFG”) in blown-colored Amadori compounds show antioxidant, antiobesity and antidiabetic effects. However, it has not been reported that the arginine derivatives of arginyl-fructose and arginyl-fructosyl-glucose could exhibit the effects of improving sexual function.

It has been confirmed by the inventor of the present invention that the arginine derivatives of arginyl-fructose and arginyl-fructosyl-glucose can promote the expression of vascular NO synthase (endothelial NOS; eNOS) and nervous NO synthase (neuronal NOS; nNOS), which are enzymes related to the generation of nitric oxide that is a main substance in erectile mechanism, and exhibit the effects in generating nitric oxide (NO) and cGMP.

Furthermore, the inventor of the present invention has developed a method for manufacturing the aforesaid arginine derivatives by using food materials from animals and plants and inducing the reaction between the sugar and the arginine contained in the food materials under a physicochemical condition.

### SUMMARY OF INVENTION

The object of the present invention is to provide arginine derivatives or salts thereof exhibiting the effects of improving sexual function, and a composition comprising them as active ingredients, for example, a medicine and a health functional food for improving sexual function.

More particularly, the object of the present invention is to provide a composition for improving sexual function comprising an arginine derivative or a salt thereof as an active ingredient, in which the arginine derivative or salt thereof does not act as a substrate for arginase, but increases the generation of nitric oxide in endothelial cells along with enhancing the expression of nitric oxide synthase and induces the increase of the content of cGMP related to an erection depending on the concentration.

Further, the object of the present invention is to provide a method for manufacturing the aforesaid arginine derivatives or salts thereof by using food materials from animals and plants which are high in arginine and inducing the reaction between the sugar and the arginine contained in the food materials under a physicochemical condition.

In addition, the object of the present invention is to establish a reaction condition for producing the aforesaid arginine derivatives or salts thereof, to provide a method for manufacturing products containing arginine derivatives based on the established condition, and to enable the utilization of the products as foods for improving sexual function.

### DETAILED DESCRIPTION OF INVENTION

In order to accomplish the above-mentioned objects, the present invention provides a reaction condition in such a manner that the sugar and the arginine contained in the arginine-containing food materials can bind with each other under a physicochemical condition when processing the food materials. In addition, the present invention provides a method for quantitatively determining the content of the arginine derivatives obtained under the reaction condition so that the content can be used as an indicator for the quality of the final product and selectiing the effective amount of the arginine derivatives. The product containing the arginine derivatives manufactured by the present invention can be provided for preventing sexual dysfunction and improving sexual function.

Hereinafter, the present invention will be described in detail.

The present invention provides an arginine derivative compound represented by the following formula (I):

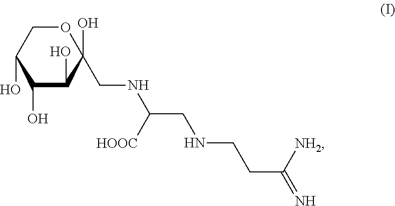

or a pharmaceutically acceptable salt thereof, wherein the compound is used for improving sexual function.

In addition, the present invention provides an arginine derivative compound represented by the following formula (II):

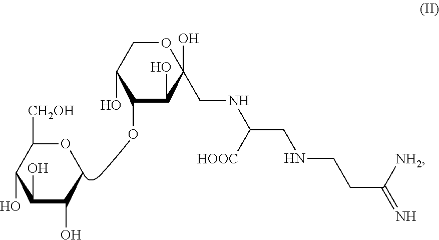

or a pharmaceutically acceptable salt thereof, wherein the compound is used for improving sexual function.

In one embodiment of the present invention, the compound of the formula (I) and/or formula (II) comprises an optical isomer, and can be present in the free form, or in the form of an acid addition salt or a base addition salt. Preferably, the acid addition salt may include hydrochloride salt, sulfate, acetate, trifluoroacetate, phosphate, fumarate, maleate, citrate or lactate, but not limited hereto.

Further, the present invention provides a composition for improving sexual function comprising at least one compound as an active ingredient selected from the group consisting of an arginine derivative compound of formula (I), an arginine derivative compound of formula (II) and pharmaceutically acceptable salts thereof:

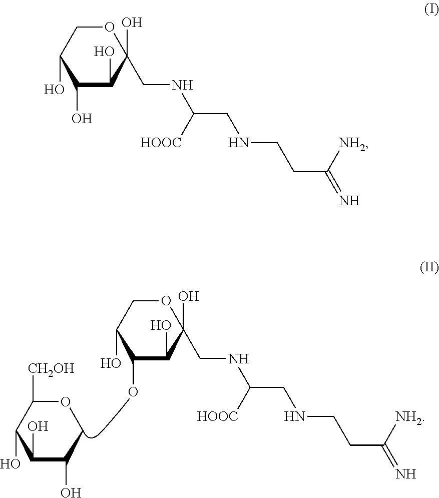

In one embodiment of the present invention, the composition for improving sexual function comprising arginine derivative compounds or pharmaceutically acceptable salts thereof derived from arginine-containing food materials can easily applied to foods. The form of the food is not specifically limited, but may include health drinks, beverages and the like. The composition of the present invention can be applied to health functional foods in the form of tea bags, capsules, powders or tablets.

In one embodiment of the present invention, the content of arginine derivative compounds or pharmaceutically acceptable salts thereof is not specifically limited in the foods. In the common foods, it is preferable to contain about 0.01 to 50 wt % of the arginine derivative compounds or pharmaceutically acceptable salts based on the weight of the final food. In the health functional foods, it is preferable to contain about 0.01 to 100 wt % of the arginine derivative compounds or pharmaceutically acceptable salts based on the weight of the final food.

In one embodiment of the present invention, the composition of the present invention may be formulated into various forms for oral administration. For oral administration, the composition may be formulated into tablets, pills, hard or soft capsules, solutions, suspensions, emulsions, syrups, granules, and elixirs, etc. Besides the active ingredients of the arginine derivative compounds or pharmaceutically acceptable salts thereof, the formulation may further include a diluent (e.g., lactose, dextrose, sucrose, mannitol, sorbitol, cellulose, and/or glycine) and/or a slip modifier (e.g., silica, talc, stearic acid and its magnesium salt or calcium salt, and/or polyethylene glycol). The tablet may further include a binder such as magnesium aluminum silicate, starch paste, gelatin, tragacanth, methyl cellulose, sodium carboxymethyl cellulose and/or polyvinylpyrrolidone, optionally a disintegrator such as starch, agar, alginic acid or its sodium salt, or a boiling mixture and/or an absorbent, a coloring agent, a flavoring agent and a sweeting agent.

In one embodiment of the present invention, the composition may be sterilized and/or may further include preservatives, stabilizers, wettable powders or emulsifiers, salts for adjusting osmotic pressure and/or as buffers, and other therapeutically useful substances. The composition may be formulated in accordance with the conventional method of mixing, granulating or coating as known in the art.

Meanwhile, garlic is used only as an example in manufacturing arginine derivatives-containing food materials since garlic is high in arginine and easily applied to the synthesis model using the reaction between L-arginine and sugar. However, the present invention is not be limited hereto but can manufacture arginine derivative compounds by using food materials derived from animals and/or plants containing arginine.

The present invention provides a method for manufacturing a composition comprising at least one compound as an active ingredient selected from the group consisting of the aforesaid arginine derivative compound of the formula (I), the aforesaid arginine derivative compound of the formula (II) and pharmaceutically acceptable salts thereof, comprising:

(a) preparing an arginine-containing food material from an animal and/or a plant;

(b) inoculating a lactic acid bacterium to a fermentation medium containing the arginine-containing food material, and fermenting the arginine-containing food material to obtain a fermented material; and

(c) heating the fermented material obtained from the step (b).

In one embodiment of the present invention, any food material from an animal and/or a plant may used as the above arginine-containing food material if it is high in arginine. Preferably, the arginine-containing food material from a plant may include ginseng, Codonopsis lanceolata, Platycodon grandiflorum, burdock, Dioscorea batatas, garlic, watermelon, legumes such as soybean, seeds such as sesame, perilla seed, pumpkin seed and sunflower seed, nuts such as peanut, almond, walnut and hazelnut, grains such as oat and buckwheat, or a mixture thereof. Preferably, the arginine-containing food material from an animal may include fishes such as salmon, puffer, carp and crucian carp, testes of fishes, intestines of fishes, shellfish such as abalone, oyster and clam, shrimp, cheese, or a mixture thereof.

In one embodiment of the present invention, the fermentation medium may contain 1 to 99 wt % of the food material such as garlic and additionally a nutrient needed for growth of the lactic acid bacterium such as casein hydrolyzate, whole milk powder, skim milk powder, soy milk powder, yeast extract, lactose, white sugar, glucose, maltose and dextrin. For example, the fermentation medium can be prepapred by mixing, pulverizing and sterilizing 1 to 90 wt % of distilled water, 3 to 80 wt % of the food material such as garlic, 0.1 to 10 wt % of dextrin and 0.1 to 10 wt % of yeast extract. Alternatively, the fermentation medium can be prepapred by mixing the pulverized food material such as garlic with the other ingredients and sterilizing the mixture.

The medium may be sterilized by a conventional method. The sterilized fermentation medium may be prepared by heating the mixture at 60 to 130° C., preferably 121° C. for 1 to 30 minutes, preferably 20 minutes under reduced pressure.

In one embodiment of the present invention, the lactic acid bacterium may be inoculated with 0.1 to 30 wt %, preferably 1 to 5 wt % into the fermentation medium, and cultured stationary or with shaking at 20 to 40° C., preferably at an optimal growth temperature of the lactic acid bacterium for 6 to 76 hours, preferably 16 to 24 hours to obtain the fermented material. In addition, pH of the fermented material is preferably adjusted to 3.0-4.0.

In one embodiment of the present invention, the fermented material may be processed by heating at 50 to 130° C., preferably 80 to 100° C. for 1 to 72 hours, preferably 2 to 8 hours to induce arginine derivatives. The resultant product may be dried by any conventional drying method such as vacuum-dried or freeze-dried, and preferably further powdered.

In one embodiment of the present invention, the lactic acid bacterium may include, for example, Lactobacillus acidophilus, Lactobacillus plantarum, Lactobacillus casei, Lactobacillus brevis, Lactobacillus delbrueckii, Lactobacillus fermentum, Bifidobacterium bifidum, Bifidobacterium longum, Bifidobacterium infantis, Streptococcus thermophilus, Leuconstoc mesenteroides, Lactococcus lactis or a mixture thereof. For example, Leuconstoc mesenteroides and Lactobacillus plantarum commonly found in kimchi, or a mixture thereof may be preferably used as the lactic acid bacterium. More preferably, Lactobacillus plantarum may be used as the lactic acid bacterium.

In addition, the present invention provides a method for manufacturing a compound represented by the following formula (II):

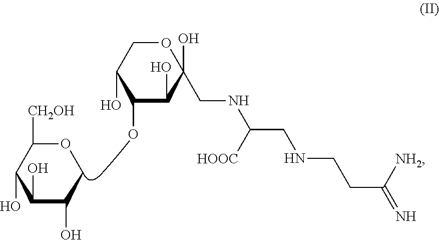

comprising, to optimally produce the compound, steaming ginseng with heating; and drying the ginseng, wherein the ginseng is repeatedly steamed and dried twice to maximize the amount of the produced compound.

In the above method, the steaming may be preferably performed at 100° C. for 25 minutes, and the drying may be preferably performed at 45° C. for 24 hours.

### ADVANTAGEOUS EFFECTS

According to the present invention, a composition for improving sexual function comprising an arginine derivative or a salt thereof as an active ingredient does not act as a substrate for arginase, but increases the generation of nitric oxide in endothelial cells along with enhancing the expression of nitric oxide synthase and induces the increase of the content of cGMP related to an erection depending on the concentration, thus remarkably exhibiting the effects of improving sexual function.

Further, a method for manufacturing the arginine derivatives or salts thereof according to the present invention involves using food materials from animals and plants which are high in arginine and inducing the reaction between the sugar and the arginine contained in the food materials under a physicochemical condition so as to establish optimum conditions for the production of the derivatives. The method of the present invention can enable the mass-production of arginine derivatives in an inexpensive manner due to the established optimum conditions, thus enabling the development of products containing arginine derivatives and the utilization of the products as foods for improving sexual function.

### BRIEF DESCRIPTION OF DRAWINGS

The above and other objects, features and other advantages of the present invention will be more clearly understood to those skilled in this arts from the following detailed description taken in conjunction with the accompanying drawings.

FIG. 1 represents chemical formulas of arginyl-fructose (AF) and arginyl-fructosyl-glucose (AFG).

FIG. 2 is a schematic diagram that shows the process for synthesizing arginine derivatives according to Preparation Example 1.

FIG. 3 a schematic diagram that shows the method for manufacturing arginine derivatives by using arginine derivatives-containing garlic according to Preparation Example 2-2.

FIG. 4 represents HPLC chromatograms of arginine derivatives of arginyl-fructose (AF) and arginyl-fructosyl-glucose (AFG) synthesized according to Preparation Example 1.

FIG. 5 represents HPLC chromatograms showing the production of arginyl-fructose (AF) derived from garlic according to Preparation Example 2-2.

FIG. 6 is a graph showing that the contents of AF and AFG contained in the processed red ginseng (Panax ginseng) vary depending on the number of heating the red ginseng according to Preparation Example 2-3.

FIG. 7 is a graph showing arginase activity for arginyl-fructosyl-glucose and arginine in Test Example 2.

FIG. 8 is an immunoblotting photograph showing the increase of the expression of eNOS and nNOS induced by arginyl-fructosyl-glucose.

FIG. 9 is a graph showing the change of the production of NO induced by arginyl-fructosyl-glucose.

FIG. 10 is a graph showing the change of the production of cGMP induced by arginyl-fructosyl-glucose.

FIG. 11 is an immunoblotting photograph showing the change of the production of the activated eNOS induced by arginyl-fructosyl-glucose.

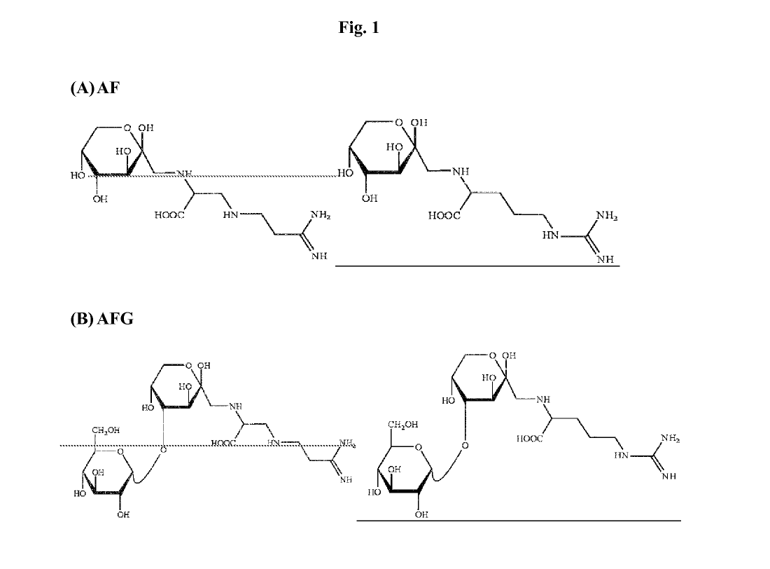

### EXAMPLES

Practical and presently preferred embodiments of the present invention are illustrated more clearly as shown in the following examples. However, it should be appreciated that those skilled in the art, on consideration of this disclosure, may make modifications and improvements within the spirit and scope of the present invention. References cited in the specification are incorporated into the present invention.

### Preparation Example 1

### Synthesis of Arginine Derivatives

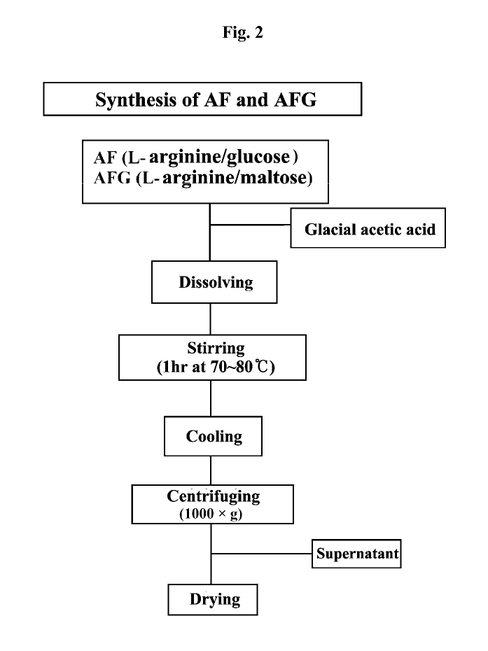

Arginyl-fructose and arginyl-fructosyl-glucose of arginine derivatives which are blown-colored Amadori compounds were manufactured by the procedures as shown in FIG. 2. For Arginyl-fructose (AF), glucose was added and dissolved into L-arginine in glacial acetic acid by mixing them. For arginyl-fructosyl-glucose (AFG), maltose was added and dissolved into L-arginine in glacial acetic acid by mixing them. Then, each of the obtained mixtures was stirred at 70˜80° C. for 1 hour. The reaction mixture was cooled to room temperature and centrifuged at 1,000×g for 10 min. The supernatant was taken and concentrated. The concentrated sample was purified through a cation exchange resin and dried. The resultant samples obtained after completing all the procedures were analyzed by HPLC and MS to confirm the purity.

### Preparation Example 2

### Preparation of Arginine Derivatives from Food Materials of Animals and/or Plants

### Preparation Example 2-1

### Culturing of Acetic Acid Bacteria

Lactobacillus plantarum (KCTC accession number 3104) which is commonly found in kimchi was incubated on agar plates (MRS agar media). One colony taken from each agar plate was inoculated into sterile culture media, and cultured stationary at 37° C. for 24 hours to obtain inoculum media. The obtained inoculum media was added to be 3% by volume in sterile culture media, and cultured under the same conditions as above to obtain the cultured strains.

In this example, Lactobacillus plantarum was used as a lactic acid bacterium, but the present invention is not limited hereto. For example, as lactic acid bacteria, the present invention can use Lactobacillus acidophilus, Lactobacillus plantarum, Lactobacillus casei, Lactobacillus brevis, Lactobacillus delbrueckii, Lactobacillus fermentum, Bifidobacterium bifidum, Bifidobacterium longum, Bifidobacterium infantis, Streptococcus thermophilus, Leuconstoc mesenteroides, Lactococcus lactis or a mixture thereof.

### Preparation Example 2-2

### Biosynthesis of AF of Arginine Derivatives from Garlic

10 wt % of garlic, 3 wt % of maltodextrin and 0.5 wt % of yeast extract were weighed and placed into a pulverizer (Hi Mixer MM-5000; Hyundai Electric Appliance Co., Ltd., Korea). 86.5 wt % of distilled water was partially added to the mixture and the mixture was pulverized in the pulverizer. The pulverized materials were transferred to a culture vessel. In addition, the remnants left in the pulverizer and the container were transferred to the culture vessel by using the distilled water. The materials in the culture vessel were heated and sterilized at 121° C. of high temperature for 15 to 20 minutes under reduced pressure, and then cooled to room temperature. The cultured strains of Lactobacillus plantarum (KCTC accession number 3104) that had been obtained according to Preparation Example 2-1 were inoculated to be 3% by volume of culture broth, and cultured stationary at 37° C. for 24 hours to obtain fermented materials derived from garlic.

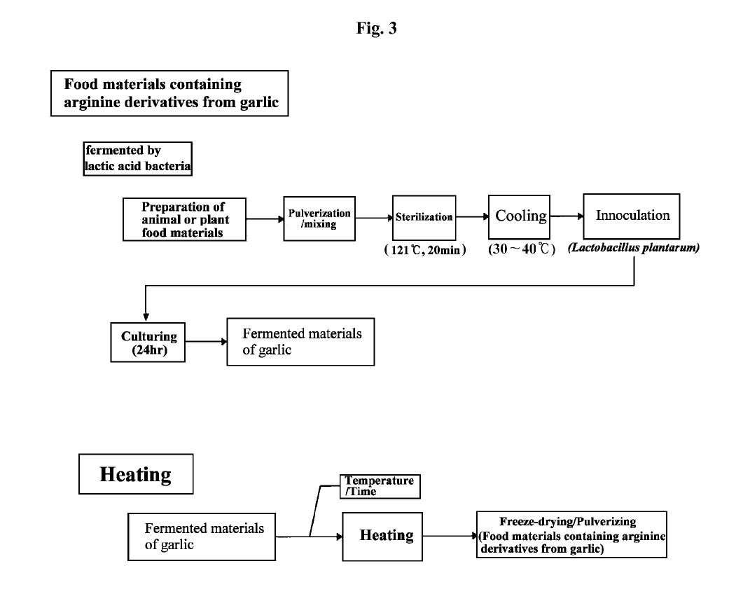

To determine conditions for producing AF from garlic, the heating was performed at 60° C., 80° C., 100° C. and 120° C., respectively by two hours. Further, the heating was also performed at 100° C. for 1 hour, 2 hours, 4 hours and 8 hours, respectively to confirm changes over time. After that, the samples were freeze-dried and then pulverized to be analyzed (see FIG. 3).

In this example, garlic was used as animal or plant food materials, but the present invention is not limited hereto. Any animal or plant food materials can be used if they are high in arginine. For example, the plant foold materials which are high in arginine may include ginseng, Codonopsis lanceolata, Platycodon grandiflorum, garlic, legumes such as soybean, nuts (peanut, almond, walnut and hazelnut, etc.), burdock, Dioscorea batatas, seeds (sesame, perilla seed, pumpkin seed and sunflower seed, etc.), watermelon, oat and buckwheat, etc. For example, the animal foold materials which are high in arginine may include shellfish such as abalone, oyster and clam, fishes such as salmon, puffer, carp and crucian carp, shrimp and cheese, etc.

### Preparation Example 2-3

### Biosynthesis of AFG of Arginine Derivatives from Red Ginseng (Panax Ginseng)

Ginseng was steamed with heat at 100° C. for 25 min and dried at 45° C. for 24 hours. The steaming and drying was repeated at least twice. 1 kg of 4 years old ginseng was washed with water and steamed for 25 minutes in a steaming pot containing 2 L of water. Then, the steamed ginseng was maintained for 60 minutes without heating and dried at 45° C. for 24 hours. Further, the steaming and drying was repeated up to nine times to obtain 9 kinds of red ginsengs which differ in the number of the steaming and drying.

### Test Example 1

### Analysis of the Contents of AF and AFG of Arginine Derivatives

For AF and AFG of arginine derivatives prepared by Preparation Examples 1, 2-2 and 2-3, the quantitative and qualitative analysis was conducted under the conditions as the following:

HPAEC-PAD system (Dionex, USA); Column—CarboPac™ PA1; Flow rate—0.7 mL/min; Temperature—30° C.; Injection—10 μl; elution time—15 minutes; eluent—360M NaOH.

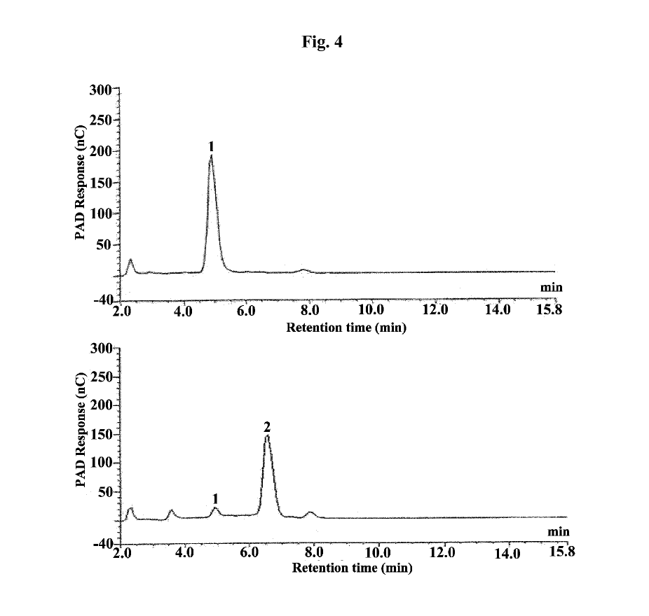

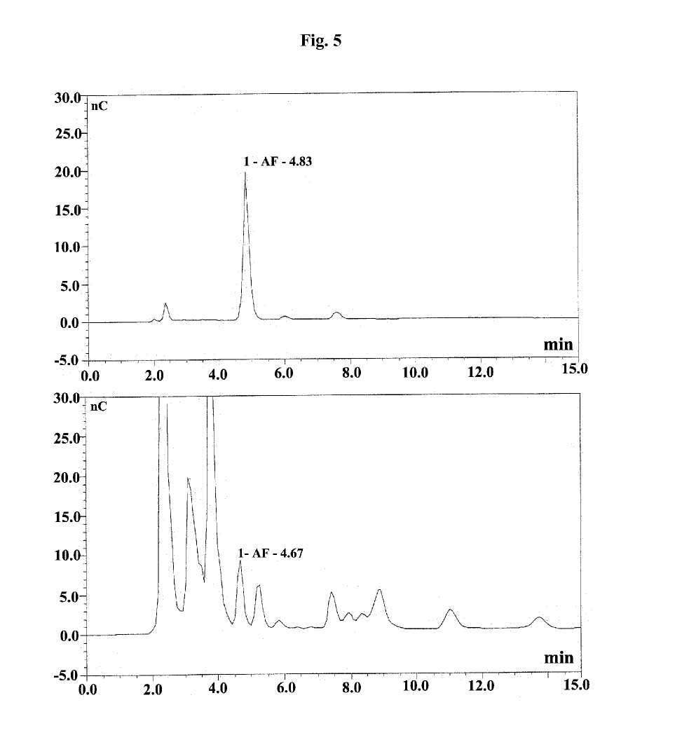

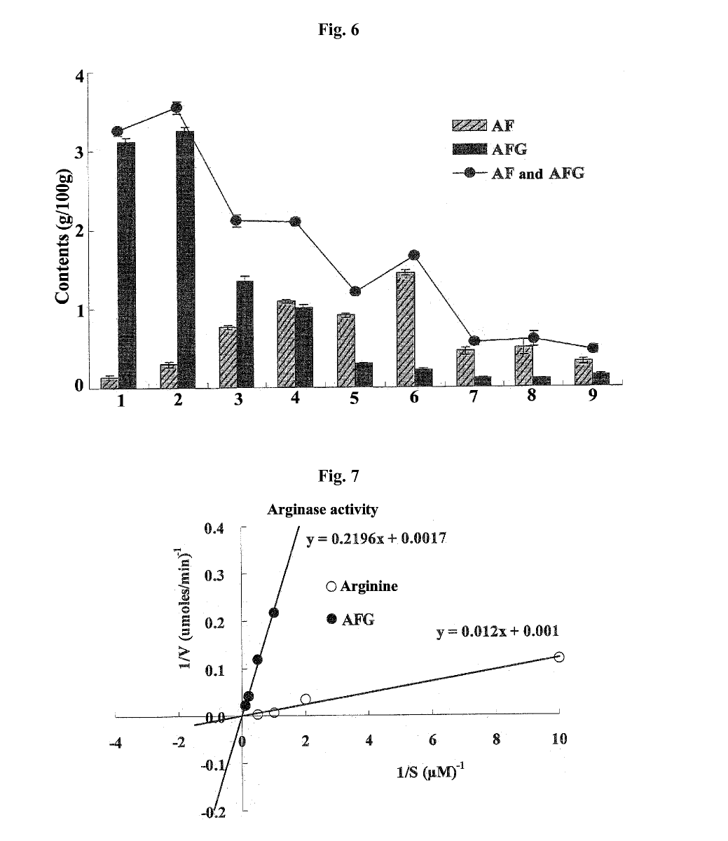

According to MS analysis, the molecular weights of AF and AFG synthesized were 337 and 449, respectively. According to HPLC analysis, the purity of AF and AFG was analyzed by 90% or more as shown in FIG. 4. Typically, AF of arginine derivatives is not detected in freeze-dried garlic and ginseng. In contrast, it was found that AF and AFG were produced in food materials derived from garlic and ginseng as manufactured by Preparation Examples 2-2 and 2-3, respectively (see FIGS. 5 and 6).

For AF of food materials derived from garlic by heating, the result of HPLC analysis was 0.46-0.82 mg/g depending on heating conditions as shown in FIG. 5. As shown in FIG. 6, it was comfirmed that the contents of AF and AFG were changed according to the method for manufacturing red ginseng from ginseng. The content of AFG is the highest in the red ginseng obtained by repeating the steaming process of red ginseng twice.

### Test Example 2

### Arginase Activity for Arginyl-Fructosyl-Glucose

20 mU arginase was dissolved in 150 μL of 0.1M carbonate-bicarbonate buffer (pH 10.0). 50 μL of L-arginine and AFG having the concentrations as shown in FIG. 7 were separately mixed with the arginase, and reacted with the arginase at 55° C. for 10 minutes. After that, 750 μL of acetic acid was added to the reaction mixture so as to stop the reaction. Then, 250 μL of ninhydrin solution was added to the mixture and was reacted at 100° C. for 1 hour. The reaction mixture was cooled and the absorbance was measured at 515 nm using ELISA reader.

A standard curve was created by using 0.01-10 μM of L-ornithine, and then the arginase activity was calculated and represented from the ornithine standard curve. As shown in FIG. 7 and in Table 1, Km values of arginase for arginine and AFG were 12 μM and 129 μM, respectively. Accordingly, it was confirmed that the decomposition rate of arginase for AFG is very low as compared with arginine.

TABLE 1
Vmax and Km of arginase for arginine and AFG

| Substrate | Vmax (μmoles/min) | Km (μM) |
|---|---|---|
| Arginine | 1,000 | 12 |
| AFG | 588 | 129 |

### Test Example 3

### Animal Experiments for Proving the Effects

8 weeks old male Sprague-Dawley rats were obtained from Orient Co., Ltd. (Korea) and used as exprimental animals. A breeding cage was conditioned with artificial lighting for 12 hours (8:00 a.m. to 8:00 p.m) and maintained at 20±3° C. The rats were provided with purified water and fed with pellet diet (Samyang diet, Korea). Food and water were not limited and supplied to the rats at a certain time of 9:00 a.m. to 10:00 a.m. every day.

### Test Example 3-1

### Administration of Samples

8 weeks old male Sprague-Dawley rats were allowed to be adapted to the environment of the breeding cage. Depending on their body weight, the rats were divided into 5 groups of control group (saline), Viagra® group (1.7 mg/kg/day), two AFG groups (200 and 400 mg/kg/day) and BGE (conventional red ginseng) group (400 mg/kg/day) and orally administered for 7 days twice a day (in the morning and in the afternoon). The amount of dietary intake was measured at a certain time of 9:00 a.m. to 10:00 a.m. every day.

### Test Example 3-2

### Taking of Tissues

After the end of feeding period, the rats were fasted for 12 hours and anesthetized with 4% hydrochloride. Then, their lower abdomens were laparotomized and their penile tissues were taken. After removing urethra from the penile tissues, the penile tissues were washed with PBS buffer solution (pH 7.2) to remove blood. The penile tissues, in which blood was removed, were rapidly freezed with liquid nitrogen and stored at −70° C. The penile tissues were stored at −70° C. before used.

### Test Example 3-3

### Measurement of the Expression of nNOS and eNOS Using Immunoblotting

After quickly pouring liquid nitrogen into the tissues and pulverizing the tissues, proteins were extracted by RIPA lysis buffer (pH 7.4) containing 50 mM Tris-HCl, 1% NP-40, 0.25% sodium deoxycholate, 150 mM NaCl, 1 mM EDTA, 1 mM PMSF, 1 mM sodium orthovanadate, 1 mM NaF, 1 mg/mL aprotinin, 1 mg/mL leupeptin and 1 mg/mL pepstatin. After quantifying the extracted proteins using Bio-Rad protein assay kit, 30 μg and 40 μg of proteins were separated by SDS-PAGE using 8% acrylamide gel for 2 hours, and transferred to nitrocellulose membrane with being blocked by 2% BSA.

The proteins to be analyzed were reacted with their primary antibodies (nNOS-1:1000, eNOS-1:500) at 4° C. for 12 hours, and the reactant was washed with TBST (pH 8.0) for 1.5 hours at intervals of 15 minutes. Then, the reactant was further reacted with secondary antibodies (rabbit 1:30000, mouse 1:15000) at room temperature for 1 hour, and washed with TBST (pH 8.0) for 1.5 hours at intervals of 15 minutes. After that, the reactant was sensitized by chemiluminescent HRP substrate (ECL) and measured by Ras-4000 mini image analyzer (Fujifilm, Japan). The density of the expressed proteins was analyzed using the program of multi gauge 3.1 (Fujifilm, Japan). As shown in FIG. 8, it was confirmed that the expressions of eNOS and nNOS were increased in the penile tissues of male Sprague-Dawley rats administered by AFG in a dose dependent manner. It was also found that the expressions of eNOS and nNOS were increased in the penile tissues of male Sprague-Dawley rats administered by the conventional red ginseng (BGE) as compared with the control group.

### Test Example 3-4

### Measurement of Nitric Oxide (NO)

The amount of the produced nitric oxide was measured by a method which slightly modifies methods proposed by Park (J. Ethnopharmacol, 2004) and Knowles (Biochem. J, 1990). After quickly pouring liquid nitrogen into the tissues and pulverizing the tissues, the pulverized tissues were mixed with 5004, of 20 mM Tris-HCl buffer solution (pH 7.2) containing 10 mM arginine, 5 μM FMN, 0.1 mM NADPH, 2004 BH₄ and 5 mM MgCl₂, and 20 μM of DAF-FM in DMSO. The mixture was incubated at 37° C. for 15 minutes and centrifuged at 10,000×g for 5 minutes, and the supernatant was used for the following analysis.

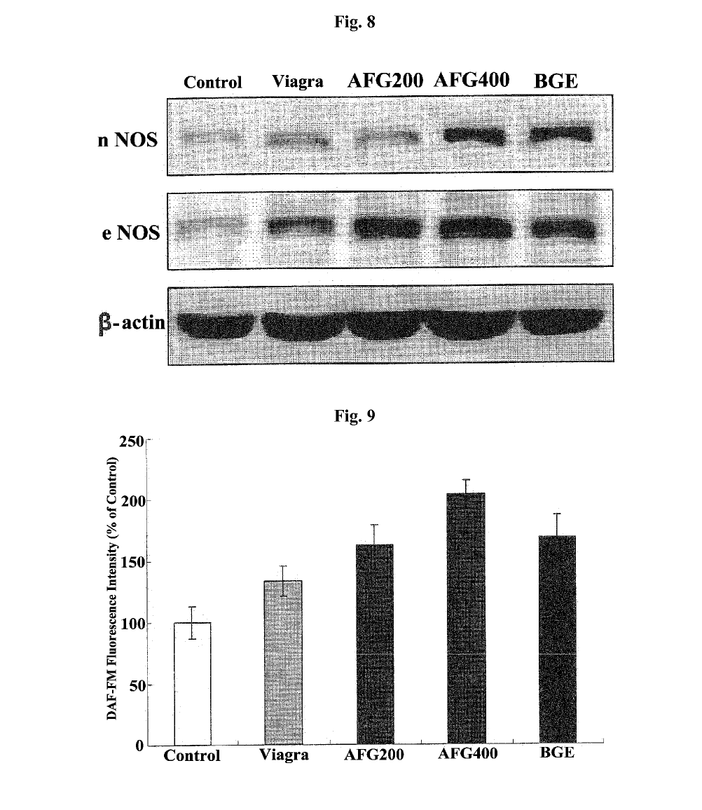

For 200 μL of the supernatant, the fluorescence intensity of DAF was measured at 485 nm of excitation wavelength and at 535 nm of emission wavelength using a fluorescence reader (TECAN Trading AG, Switzerland). As shown in FIG. 9, it was confirmed that the production of nitric oxide (NO) was increased in the penile tissues of male Sprague-Dawley rats administered by AFG in a dose dependent manner as compared with the control group. It was also found that the production of nitric oxide was increased in the penile tissues of male Sprague-Dawley rats administered by the conventional red ginseng (BGE) as compared with the control group.

### Test Example 3-5

### Measurement of cGMP

The concentration of cGMP was measured by a method which slightly modifies methods proposed by Park (J. Ethnopharmacol, 2004) and Knowles (Biochem. J, 1990). After quickly pouring liquid nitrogen into the tissues and pulverizing the tissues, the pulverized tissues were treated with 20 μM zaprinast dissovled in 20 mM Tris-HCl buffer solution (pH 7.2) containing 0.1 mM CaCl₂ at 37° C. for 2 hours. The treated tissues were reacted with 5004, of 20 mM Tris-HCl buffer solution (pH 7.2) containing 10 mM arginine, 504 FMN, 0.1 mM NADPH, 20 μM BH₄, 5 mM MgCl₂, 5 mM GTP, 1 mM 3-isobutyl-1-methylxanthine, 0.75 mM DL-DTT(DL-dithiothreitol) at 37° C. for 15 minutes, and then centrifuged at 10,000×g for 5 minutes, and the supernatant was used for the following analysis.

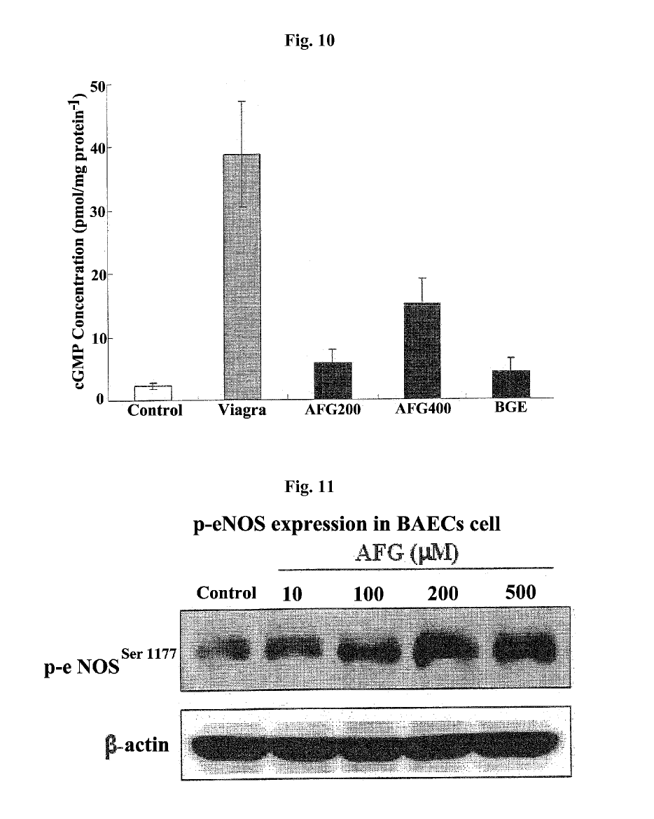

A standard curve was created by using 0.8-50 pmol/mL of cGMP, and the concentration of cGMP was measured by cGMP ELISA kit (Aassay Designs, Inc., Michigan). As shown in FIG. 10, it was confirmed that the concentration of cGMP was increased in the penile tissues of male Sprague-Dawley rats administered by AFG and the conventional red ginseng (BGE) as compared with the negative control group. However, it was also found that the increase of the concentration of cGMP induced by AFG and the conventional red ginseng (BGE) was less than the positive control group of Viagra®. This phenomenon can be explained by the result that Viagra® acts as an inhibitor of phosphodiesterase (PDE-5) that is a hydrolase of cGMP and thus the concentration of cGMP becomes too high.

The maintenance of the high concentration of cGMP makes the erection time of penis be unnecessarily longer to cause side effects of Viagra®. Accordingly, it was confirmed that when fed by AFG of arginine derivatives, the expression of NOS and the production of NO were promoted in the penile tissues, and thus AFG could help to prevent the erectile dysfunction and premature ejaculation.

### Test Example 4

### Expression of Activated eNOS in Endothelial Cells

Endothelial cells (BAECs) previously treated with the samples were washed with cold PBS buffer (pH 7.2) twice and then treated with RIPA lysis buffer [50 mM Tris-HCl, 150 mM NaCl, 0.5% sodium deoxycholate, 1% NP 40, 1% phosphatase inhibitor cocktail, 1 mM PMSF] in 150 μL/well to collect cell extracts. The protein content of the collected cell extracts was determined using Bradford assay, and then 25 μg of the protein was separated by using SDS-PAGE and transferred to nitrocellulose membrane. The nitrocellulose membrane was blocked with 1% BSA (Bovine serum albumin) solution at room temperature for 1 hour, and reacted with a primary antibody specific for the target protein at 4° C. for 12 hours. Upon completion of the reaction, the nitrocellulose membrane was washed with TBST (10 mM Tris-HCl, 150 mM NaCl, 0.05% NP 40) several times, and reacted with a secondary antibody binding to HRP (Horse radish peroxidase) at room temperature for 1 hour. After that, the nitrocellulose membrane was washed again with TBST and the target protein was confirmed by using ECL solution. To identify the target protein, luminesent image analyzer (LAS 4000 mini) were used. Two kinds of eNOSs are present in endothelial cells as an active form and an inactive form. It is possible to produce nitric oxide (NO) when eNOS is phosphorylated to be activated. According to FIG. 11, it was confirmed that the expression of the activated eNOS was increased in the endothelial cells (BAECs) by AFG in a dose dependent manner.

As mentioned above, it was confirmed that AF and AFG were produced using arginine derivatives-containing food materials such as food materials derived from garlic in manufacturing arginine derivatives based on AF and AFG synthetic model of the arginine and the sugar, according to the present invention. In addition, the effects for improving sexual function induced by the arginine derivatives were confirmed through in vitro and in vivo experiments using AFG. Thus, the arginine derivatives-containing food materials prepared by the present invention can improve sexual function, and thus will be useful as a variety of related products or their raw materials.

Although the present invention has been illustrated and described with reference to the exemplified embodiments of the present invention, it should be understood that various changes, modifications and additions to the present invention can be made without departing from the spirit and scope of the present invention.

1. An arginine derivative compound represented by the following formula (I): 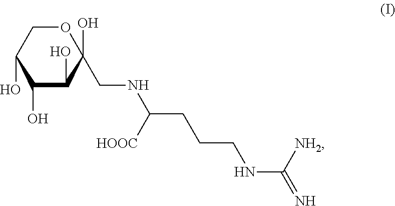
or a pharmaceutically acceptable salt thereof, wherein the compound is used for improving sexual function.

2. An arginine derivative compound represented by the following formula (II): 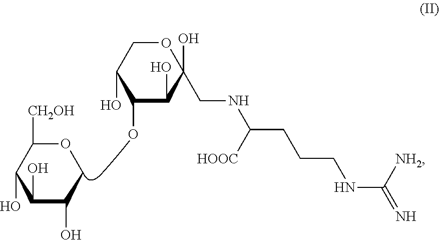
or a pharmaceutically acceptable salt thereof, wherein the compound is used for improving sexual function.

3. The arginine derivative compound as claim 1, wherein the compound comprises an optical isomer, and can be present in the free form, or in the form of an acid addition salt or a base addition salt.

4. A composition for improving sexual function comprising:
at least one compound as an active ingredient selected from the group consisting of an arginine derivative compound represented by the following formula (I); 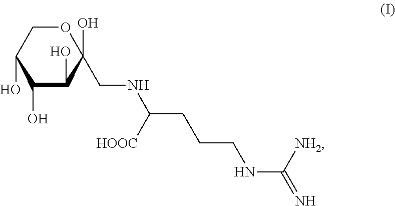
an arginine derivative compound represented by the following formula (II); 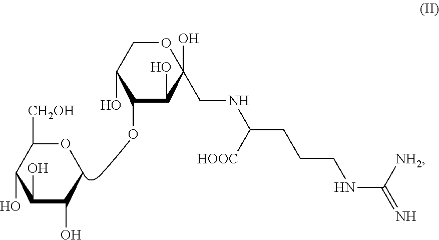
and; pharmaceutically acceptable salts thereof.

5. The composition as claimed in claim 4, wherein the composition is formed in the food form such as health drinks, beverages, tea bags, capsules, powders or tablets.

6. The composition as claimed in claim 4, wherein the composition is formulated into tablets, pills, hard or soft capsules, solutions, suspensions, emulsions, syrups, granules, or elixirs.

7. A method for manufacturing a composition comprising at least one compound as an active ingredient selected from the group consisting of an arginine derivative compound represented by the following formula (I), an arginine derivative compound represented by the following formula (II) and pharmaceutically acceptable salts thereof, comprising: 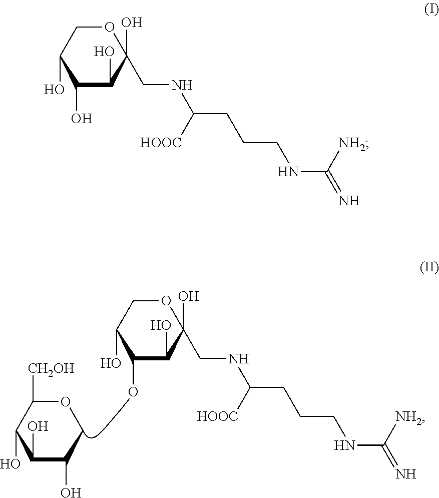
(a) preparing an arginine-containing food material from an animal and/or a plant;
(b) inoculating a lactic acid bacterium to a fermentation medium containing the arginine-containing food material, and fermenting the arginine-containing food material to obtain a fermented material; and
(c) heating the fermented material obtained from the step (b).

8. The method as claimed in claim 7, wherein the arginine-containing food material is selected from the group consisting of ginseng, Codonopsis lanceolata, Platycodon grandiflorum, burdock, Dioscorea batatas, garlic, watermelon, legumes, seeds, nuts, grains, fishes, intestines and testes of fishes, shellfish, shrimp, cheese and a mixture thereof.

9. The method as claimed in claim 7, wherein the fermented material is processed by heating at 50 to 130° C. for 1 to 72 hours.

10. The method as claimed in claim 9, wherein the fermented material is processed by heating at 80 to 100° C. for 2 to 8 hours.

11. The method as claimed in claim 7, wherein the product obtained by performing the step (c) is vacuum-dried or freeze-dried and powdered.

12. The method as claimed in claim 7, wherein pH of the fermented material is adjusted to 3.0-4.0 at the step (b).

13. The method as claimed in claim 7, wherein the lactic acid bacterium is at least one selected from the group consisting of Lactobacillus acidophilus, Lactobacillus plantarum, Lactobacillus casei, Lactobacillus brevis, Lactobacillus delbrueckii, Lactobacillus fermentum, Bifidobacterium bifidum, Bifidobacterium longum, Bifidobacterium infantis, Streptococcus thermophilus, Leuconstoc mesenteroides and Lactococcus lactis.

14. The method as claimed in claim 13, wherein the lactic acid bacterium is Lactobacillus plantarum.

15. A method for manufacturing a compound represented by the following formula (II): 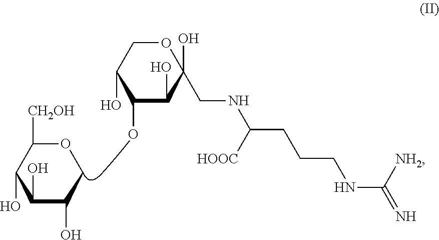
comprising steaming ginseng with heating; and drying the ginseng,
wherein the ginseng is repeatedly steamed and dried twice to maximize the amount of the produced compound.

16. The method as claimed in claim 15, wherein the steaming is performed at 100° C. for 25 minutes, and the drying is performed at 45° C. for 24 hours.
